(19) United States
(12) Patent Application Publication
(10) Pub. No.: US 2007/0265630 A1
(43) Pub. Date: Nov. 15, 2007

(54) Fixing Device for Clamping the Ends of a Surgical Cable Used for Fixing Bone Parts

(75) Inventors: Roelof Marissen, Born (NL); Leonard J.A. Nielaba, Eygelshoven (NL); Willibrordus M.E. Theunissen, Bocholtz (NL)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 10/586,496

(22) PCT Filed: Jan. 27, 2005
(86) PCT No.: PCT/EP05/00935

§ 371 Date: Sep. 6, 2006

(30) Foreign Application Priority Data
Jan. 30, 2004 (EP) 04075278.4

(51) Int. Cl. A61B 17/82

(52) U.S. Cl. 606/74

(57) ABSTRACT

Fixing device, comprising a surgical cable (22) having a first and a second free end and at least three fixing plates (2, 4, 6) positioned in a stack as a top plate (6), a middle plate (4) and a bottom plate (2), leaving a gap (24, 26) between two adjacent plates, each fixing plate having a ring (14, 16, 18) surrounding a central hole (8, 10, 12), the rings having an inner edge adjacent to the hole and an outer edge along a circumference of the fixing plate, wherein the holes in the stacked position at least partly overlap each other thus forming a central hole in the stack, and wherein at least one working end of the cable follows a specified continuous trajectory containing sections running through the hole, around the outer edges and underneath, over and between the rings of the plates.

The invention relates to a fixing device for tying together objects, in particular for fixing bone parts by means of a cable, in particular a surgical cable.

In modern surgery on many occasions there is a need for pressing together and preventing relative movements of bone parts that have been separated in the course of an operation and have to grow together again or to keep a bone part at a fixed and constant distance and position with respect to another bone part or an orthopedic device such as a splint, which actions are further denoted as fixing bone parts. Also in the treatment of bone fractures this need for fixing bone parts that came apart is required for at least the time needed to have the body repair the fracture or for longer times, in many cases even for years.

It is known to wrap a steel cable around the bone parts to be fixed, to bring the cable under the required tension to fix the parts against relative movement, e.g. Under load, and to leave it in place at least until the bone parts have grown together and the bone has recovered sufficiently to take up its proper function again, or even permanently to avoid a further operation to remove the cable. The cable is tensioned and fixed by guiding its ends from opposite sides through holes in a metal block, tensioning the cable by exerting a drawing force on the ends and pinching the metal block such that the holes collapse and fix the cable.

The use of steel cables brings about a number of disadvantages. They are prone to fatigue leading to breakage of the composing steel fibers after which the sharp ends stick out into the body. Breakage of the fibers during their application by a surgeon brings the risk of stitching and possible blood contact. Further, steel is a hard material and being tensioned around the bone there is the risk of carving of the steel cable into the bone.

U.S. Pat. No. 5,540,703 teaches to use instead of a metal cable a braided polymeric material cable and to lock the tightened and tensioned cable with non-loosening knots, in order to overcome certain disadvantages of metal cables. High performance, i.e. high strength, high modulus, polyethylene in particular is applied as the polymeric material. Fibers of this type, however, are notorious for their difficulty to be fixed by knots, clamps or other means when they are under tension.

The present invention now seeks to provide a method and means for fixing bone parts by means of a surgical cable of a polymeric material that do not suffer from the disadvantages of the known means and cope with the fixing difficulties related to the application of tensioned high performance fibers.

This aim is achieved according to the invention by supplying a fixing device according to claim 1.

It is understood that the trajectory has been defined in the situation that the plate are in vertically stacked position. Other situations will be rotated and/or mirrored situations but in these situations the shape and the order of the parts in the trajectory remain essentially the same and can be easily translated to a vertically stacked position.

The novel fixing device can be easily applied to keep in place bone parts by applying the cable around the parts to be fixed and drawing the ends of the cable that at the end of the trajectory specified thrust out of the fixing plates. It takes a relatively small force to tension the cable by drawing the two working ends, which at the same time enlarges the clamping force of the two plates on the cable part that runs between the rings of the plates. This clamping, fixing force has appeared considerably larger than the force exerted by the tensioning of the cable by pulling the working ends.

In the method according to the invention a fiber surgical cable having two ends is applied. The fiber is a high performance fiber, preferably a polyethylene fiber having a tensile strength of at least 1.8 GPa and a modulus of at least 60 GPa. Examples of such fibers are various Dyneema grades of DSM High Performance Fibers and various Spectra grades of Honeywell Inc. These fibers have been prepared from high molecular weight polyethylene, in particular polyethylene heaving a weight average molecular weight of at least 2,000,000.

In particular the cable is a bundle of parallel, twisted or braided fibers of the type described above. It may also be a high performance tape having the required strength and modulus. The tape may be a single tape or it may be in the form of a flat braid of high performance fibers. Twisting and braiding are commonly applied techniques in cable production and cables obtained by these common techniques are applicable in the device according to the invention. It should be noted that in constructions of these fibers, e.g. in braids and twisted bundles an efficiency loss occurs, i.e. that the resulting strength of the construction is lower that then the sum of the strengths of the constituting fibers. The efficiency depends on the used braid construction, braiding period and braiders. Braid efficiency may range from 30-70%. Starting from the required strength of the cable in each case the proper combination of initial fiber strength, cable thickness and cable construction can be chosen to obtain a cable having at least that required strength. The forces required to fix bone parts generally range from 500 to 3000 N, depending on the size of the bones to be fixed and the forces exerted on the bone parts. For small bones, like in fingers, smaller forces and thickness may be relevant. In general the total thickness of the cable will range from 500 to 30,000 dtex.

The cable must be suited to be positioned around bone parts and has an oblong shape; in particular the cable is a bundle of parallel, twisted or braided fibers of a length that is sufficient to be laid around bone parts to be fixed and to be tensioned.

The cable in the described shape of a bundle of fibers has two working ends. These ends normally will have been treated to prevent unraveling or splitting of the bundle. The ends can e.g. have been treated with a substance gluing together the fibers, have been molten together or otherwise be prevented from unraveling. In this embodiment the last few centimeters of the bundle to the ends form the end parts. In certain embodiments of the invention one end of the fiber may have been formed into an eye by splicing the end back into the bundle.

The device comprises three fixing plates. Here and hereafter a fixing plate is understood to be a flat or slightly curved piece of a material that can withstand the tensile forces specified herein before that are required in bone fixing and is biocompatible. Examples of suitable materials are reinforced thermoset resins, metals and ceramic material. A material is considered biocompatible if it is tolerated when implanted into the human or animal body without causing pain, inflammation, irritation, and poisoning or other unwanted effects to the human and animal body.

The fixing plate mainly is flat but it may be slightly curved to be in conformity to a bone part it will be in contact with.

Each fixing plate comprises a central hole and a ring surrounding the hole. The central hole preferably encompasses the center of the fixing plate but this center is not necessarily the center of the hole. The hole may be circular, oval, square or rectangular or any other regular smooth shape. The same holds for the outer circumference of the ring surrounding the hole. The shape of the hole and of the outer circumference may be the same or different. The ring has an inner edge adjacent to the hole and an outer edge forming the outer circumference of the ring. Preferably the edges are rounded in order to prevent cutting or stitching effects to the surrounding tissues after being applied into a human or animal body and to prevent total or partial cutting of the surgical cable both during application and afterwards.

The size of the fixing plates is chosen to match both the strength requirements and the size requirements set by the size and shape of the bone part it will be applied to. The ratio of the largest dimension of the hole and the largest width of the ring, i.e. the distance between the inner and the outer edge of the ring may vary over a large range and may lie between 0.3 and 0.9. Generally the thickness will be in the range from 0.5 to 4 mm and the largest dimension of the ring will be in the range of 4 to 30 mm. Thus the fixing plates may have the shape of a chain link on the one end of the scale, in which the hole is relatively large and the ring has a relatively large thickness to width ratio, to the shape of a washer on the other end of the scale, in which the hole is relatively small and the ring has a relatively small thickness-to-width ratio. Preferably the thickness of the plates is not larger than 0.5 times to even lower than 0.1 times the largest width of the ring in order to provide a stable positioning of the plates on top of one another. Outer and inner edges of the rings are preferably rounded in order to prevent cutting from the surgical cable at sharp edges when it is tensioned. The radius of these rounded edges preferably is between 0.2 and 2 mm.

The three fixing plates are in a stacked position, leaving a gap between each two adjacent rings. The width of this gap initially will be about the thickness of the surgical cable. After the cable has been tensioned the fixing plate will be pressed together by the forces exerted for tensioning thus preventing the cable form slipping loose.

The holes at least partly overlap each other when the plates are stacked in a centered manner thus forming a central hole in the stack. Preferably the three plates are of equal shape and size at least with respect to the parts clamping the cable and also preferably the holes have the same size and position within the plate.

The three plates are connected to the cable in a specific way, resulting in a device that can be easily tensioned and in a tensioned state is capable to retain the tension without the cable sliding back. To achieve this at least one free end of the cable has to follow
(1) a first continuous trajectory starting from outside the stack running consecutively
(a) underneath the stack along the bottom side of the ring of the bottom plate from its outer edge to its inner edge,
(b) through the central hole along the inner edge of the rings of the bottom plate, the middle plate and the top plate respectively to a position above and outside the stack,
(c) over the top side of the ring of the top plate from its inner edge to its outer edge,
(d) along the outer edges (I) of the ring of the top plate, (II) of the rings of the top and the middle plate or (III) of the rings of the top, the middle and the bottom plate to a position, in case (I), at the level of the gap between the top and the middle plate, in case (II), at the level of the gap between the middle and the bottom plate and in case (III), below the stack,
(2) a second continuous trajectory, comprising in non-prescribed order and directions at least a section (e) through the gap between the top and the middle plate and a section (f) through the gap between the middle and the bottom plate, the second trajectory starting in case (I) with section (e), in case (II) with section (f) and in case (III) with a section (g) running along the bottom side of the ring of the bottom plate, the second trajectory further comprising one or more connection sections, running through at least a part of the central hole or outside the outer edge of one or more of the rings, to make the trajectory continuous.
   The cable between the free ends can surround e.g. bone parts to kept in a mutually fixed position. By pulling the free ends the cable is tensioned and due to the specific trajectory the ends follow through the stack of fixing plates these plates are pressed together thus preventing the cable ends from slipping back. This results in the e.g. bone parts being firmly fixed due to the tension in the cable.

With the defined trajectory running as (e) and (f) through both gaps between the two adjacent ring pairs (top-middle and middle-bottom) the cable is clamped at two places and due to the presence of the trajectory parts (a) and (c) the whole stack is pressed together when the cable is tensioned.

It was found that less tensioning force is required when the second trajectory end s with a last section being one of the sections (e) or (f). It was further found that tensioning is easier when the free end ends outside the stack rather than in the central hole and from there is brought to outside the stack for being grabbed to be tensioned. Thus preferably the cable in said last section runs through the corresponding gap from the inner edge to the outer edge of the rings adjacent to that gap bringing the free end from the central hole to outside the stack.

An even higher clamping force was found to be obtained when the first trajectory runs along both the outer edges and the inner edges of all three rings. The first trajectory then runs in the order (a), (b), (c), (d) and together with the, in this case (III), first section (e) of the second trajectory the end of the cable forms a complete loop around the stack. This effect is further enhanced when the second trajectory further comprises a section (h) running over the topside of the ring of the top plate.

In general both free ends of the cable may follow one of the trajectories defined above. Since the clamping force to a certain extent may hamper ongoing tensioning of the cable by pulling the free ends of the cable, in a preferred embodiment only one end of the cable follows the defined trajectory whereas the other end is connected to a tensioning device connected to the fixing plates. Thus after maximally tensioning the cable by drawing the one cable end a further tensioning can be obtained by operating the tensioning device. Such tensioning device may comprise a mechanism as used in a turn buckle, a worm wheel and driving screw combination or two cooperating 45° tooth wheels rotating around mutually perpendicular axes or even a simple screw, screwed in the tensioning device and ending in a hook that can be connected to the other end of the cable by a knot or hooking to an eye at that end. These tensioning devices can be connected to the cable in such a way that only a drawing force is exerted on the cable, resulting in its shortening and tensioning but also in such a way that, instead of or next to the drawing force, also a twisting force is exerted the cable, also resulting in further tensioning the cable. When such a device is used the other end of the cable may be knotted to e.g. a hook of the tensioning device but preferably the other end of the cable contains an eye made e.g. by a split or any other non-slipping means for connecting it to the tensioning device.

In a preferred embodiment in one of the two rings adjacent to the gap between the plates through which the last part of the second trajectory runs in its surface facing said gap a continuous groove is present running between the outer edge and the inner edge of said ring and in the other ring adjacent to said gap in its surface facing said gap a ridge is present matching with said groove. Such ridge-groove combination may also be present in both pairs of adjacent plates. The middle plate than may have a ridge on both sides, a groove in both sides or a groove in one side and a ridge on the other, whereas the top and bottom plate then in each case have a matching groove or ridge. This embodiment is best applicable when both ends of the cable follow a trajectory around and between the plates as described above and later.

Groove and ridge are continuous which means that they run as a uninterrupted loop surrounding the hole. Ridge and groove are matching in the sense that their respective positions and dimensions are such that when they are placed on top of one another the ridge falls in the groove and fits without leaving virtually any space between the two facing ring surfaces, both on the ring and in the ridge-groove pair. The space left between ridge and groove should be equal to or smaller than the space between the two ring surfaces outside the ridge and groove. This shape brings the advantage that the tensioned cable is clamped to the highest extent between the ridge and the groove. This shape brings the advantage that the tensioned cable is even better fixed and secured against slipping.

In case the shape of the fixing plates is asymmetric and one can predict with sufficient certainty which position the fixing plates will take when the surgical cable is tensioned, in another embodiment of the invention the matching ridge and groove can be present at only the part of the rings where the cable will run when the fixing plates are in said predicted position.

In yet another embodiment the invention relates to a device in which one end of the cable follows a trajectory as described before whereas the other end is fixed to a tensioning device that is connected to the fixing plates and one of the two rings adjacent to the gap between the plates through which the last part of the second trajectory runs in part of its surface facing said gap a groove is present running between the outer edge and the inner edge of said ring and in the other ring adjacent to said gap in its surface facing said gap a ridge is present matching with said groove.

In this embodiment the groove and ridge need only be present at the side of the plate where the one cable end is connected to the stack of plates. At the opposite side where the fixing device is present groove and ridge have no function and need not be present here.

The longitudinal shape of the ridge and the groove need not be the same as, but preferably largely follows the contour of the inner and/or outer-edge of the ring. Preferably they follow a smooth trajectory, i.e. with rounded curves having a radius of at least 0.1, preferably at least 0.3 and even at least 0.5 mm in order to prevent the risk of unwanted cutting of the cable at sharp edges. The height of the ridge and accordingly the depth of the matching groove are preferably at least 30% and at most 90% of the thickness of the fixing plate. More preferably this height lies between 40 and 75% of said thickness. The depth of the groove should be such that the strength of the plate is such that the plate can withstand the drawing forces on tensioning of the cable. The width of the ridge at its base follows from its height and top angle as defined hereafter. These are preferably chosen such that said width does not exceed 95% of the distance between the outer edge and the inner edge of the ring. The position of the centerline of ridge and groove is not critical and they may be equally spaced from the inner and outer edge of the ring but also may be located within 10 and 90% of the local width of the ring there from. The ridge may have the shape of a triangle with a top angle of between 45 and 1200, preferably between 60 and 1050 and preferably has a rounded top having a radius of between 0.1 and 0.3 mm. The rounding radius will usually be rather small, in order to keep as much surface of the ridge as possible, but cutting of the fibers by a too sharp edge must be prevented.

The invention further relates to a method for tying objects together, in particular for fixing bone parts comprising the steps of applying a fixing device wherein the two cable ends follow a trajectory as described here before around the objects to be tied together, in particular the bone parts to be fixed, followed by drawing the ends of the cable to tension the cable around the objects to be tied together, in particular the bone parts to the tension required to tie the objects together, in particular to fix the bone parts.

In a preferred embodiment of this method a bar is inserted between one or both sets of adjacent fixing plates before the cable is tensioned and is removed after the cable has at least partly been tensioned. This embodiment has the advantage that the clamping force only works after the bar has been removed and is not hampering the tensioning of the cable before. The thickness of the bar can be small but to have the full benefit of its presence it is preferably at least equal to the thickness of the cable. Since the removal of the bar may lead to a minimal elongation of the tensioned part of the cable the bar should be thin, preferably no more than 5 and more preferably no more than 2 or even 1 mm.

In another embodiment the invention relates to a method for tying objects together, in particular for fixing bone parts comprising the steps of applying around the objects to be tied together, in particular the bone parts to be fixed a fixing device wherein one end of the cable follows a trajectory as described here before and the other end is fixed to a tensioning device that is connected to the fixing plates, followed by drawing said one end of the cable to tension the cable around the objects, in particular around the bone parts, removing the slack in the cable, and then tensioning the cable to the tension required to tie the objects together, in particular to fix the bone parts by operating the tensioning device.

From the drawings the information can be taken as how to lead the cable along the proper trajectory around the fixing plates.

The various embodiments and methods described are not only suitable for tying together objects or for the fixing of bone parts but they are also useful for the connection of bones to artificial elements providing some orthotic function, e.g. a splint. Where the wording ‘bone parts’ is used this is considered also to encompass one bone part and an artificial element that need be tied together or fixed.

The invention also relates to essential parts of the fixing device, in particular the invention further relates to a set of at least three fixing plates and a surgical cable, to a surgical cable and to a plate, prepared or fitted for constructing a fixing device according to the invention or prepared for application in the method of the invention. In a preferred embodiment the invention relates to a set of two fixing plates, in the ring of one of the plates in its surface a continuous groove being present running between the outer edge and the inner edge of said ring and in the ring of the other plate on its surface facing the gap a ridge being present matching with said groove.

The invention will be further illustrated by the following drawings, of which

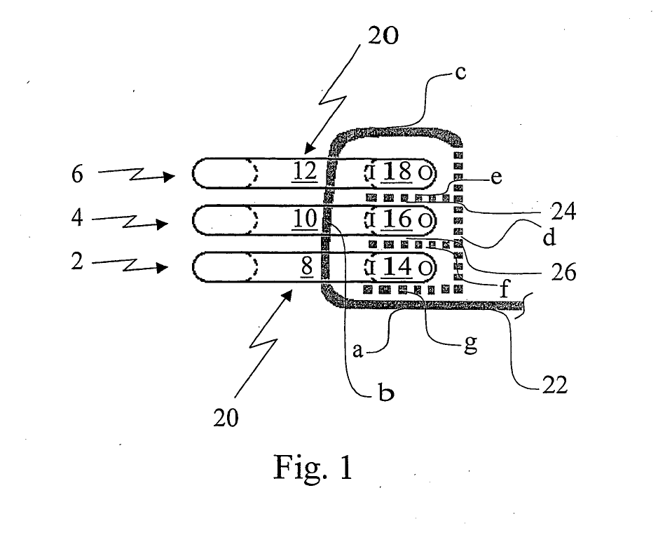

FIG. 1 is a schematic side view of the fixing device according to the invention showing the essential parts of the cable trajectory;

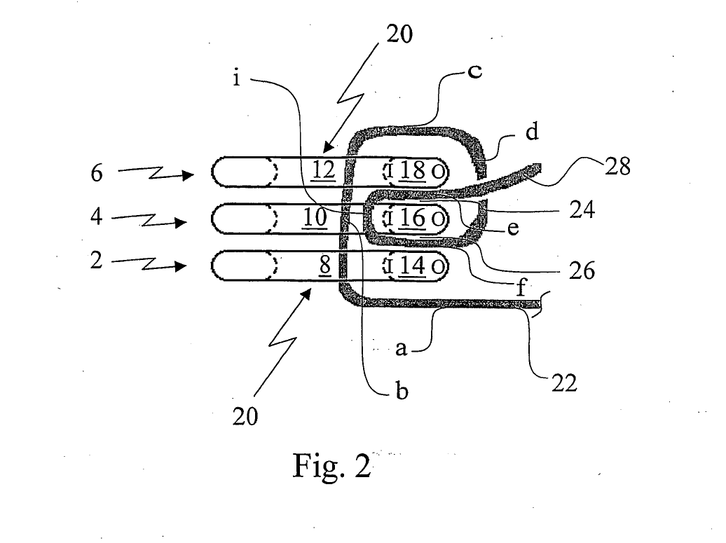

FIG. 2 is a schematic side view of a first embodiment of the fixing device according to the invention;

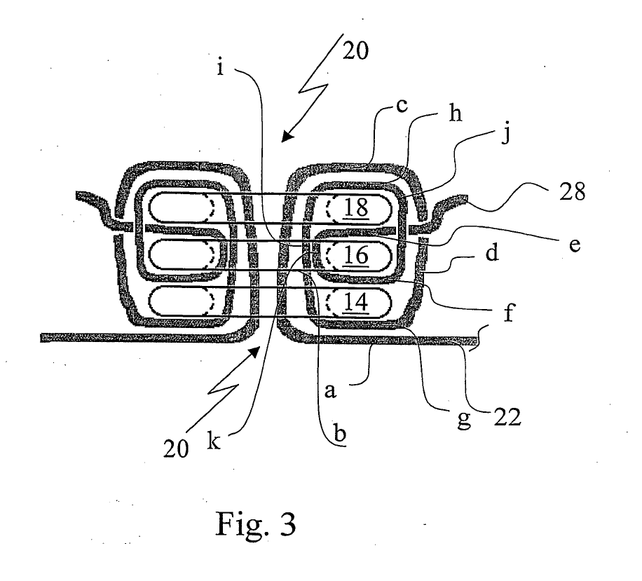

FIG. 3 is a schematic side view of a second embodiment of the fixing device according to the invention;

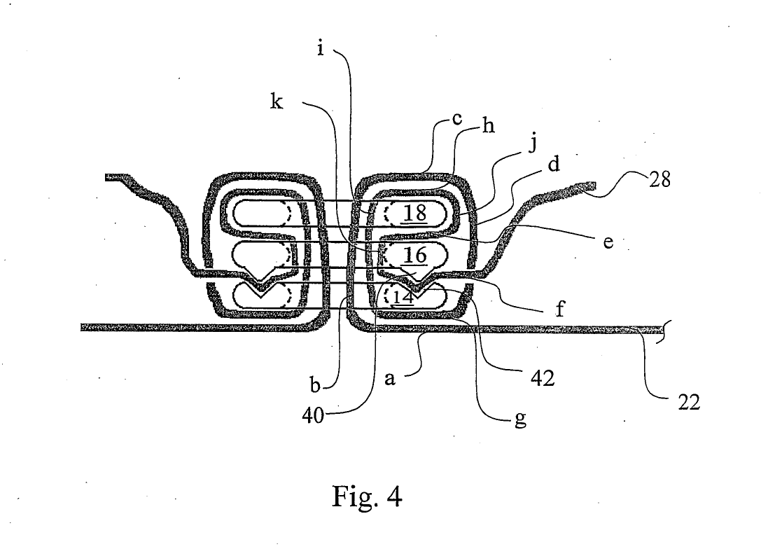

FIG. 4 is a schematic side view of a third embodiment of the fixing device according to the invention;

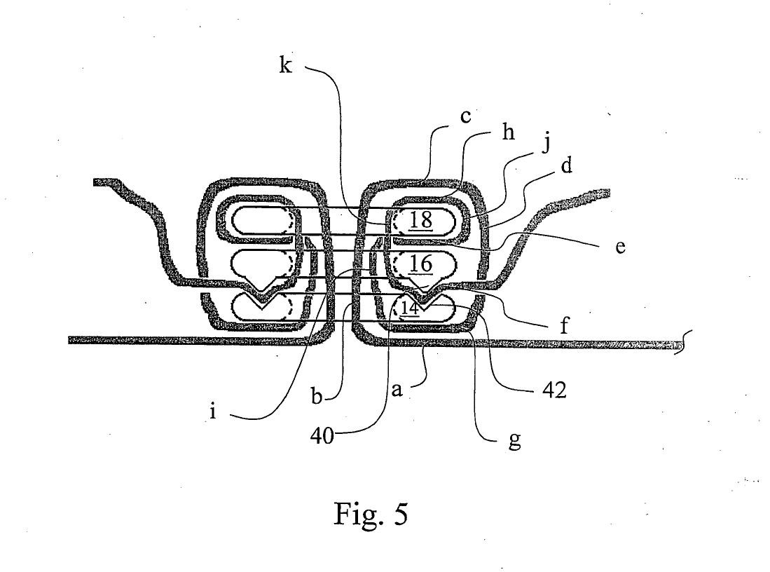

FIG. 5 is a is a schematic side view of a forth embodiment of the fixing device according to the invention;

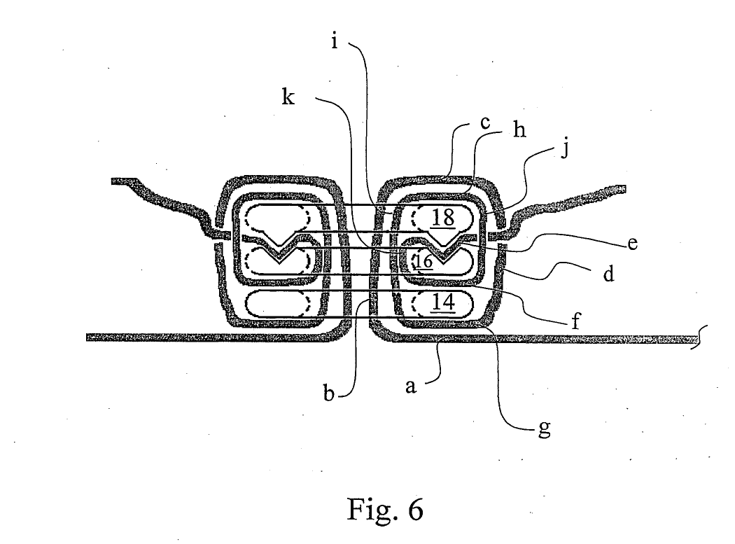

FIG. 6 is a schematic side view of a fifth embodiment of the fixing device according to the invention;

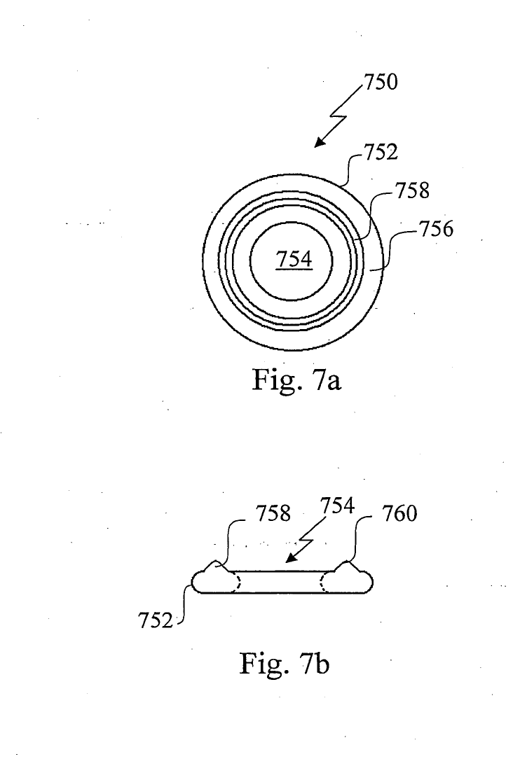

FIG. 7a is a top view of a fixing plate according to the invention bearing a ridge;

FIG. 7b is a side view of this fixing plate of FIG. 7a;

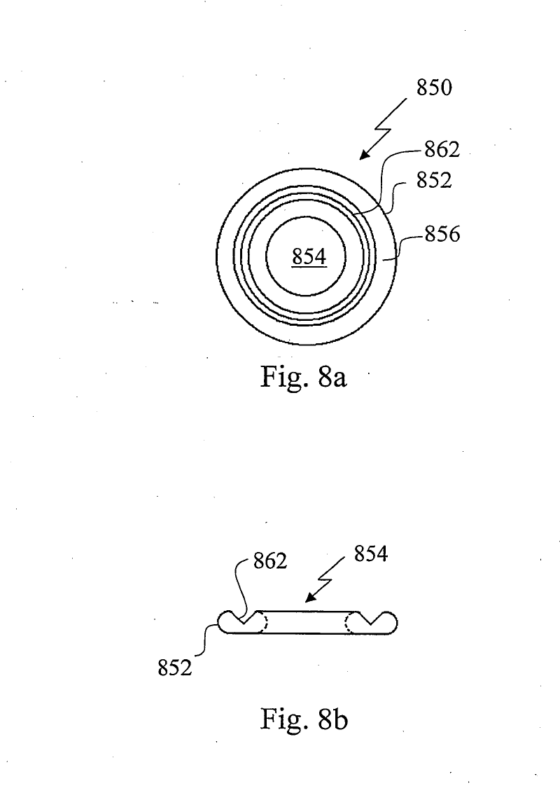

FIG. 8a is a top view of a fixing plate according to the invention having a groove and being matching with the fixing plate of FIG. 7a; and

FIG. 8b is a side view of this fixing plate of FIG. 8a.

In FIG. 1 the numbers 2, 4 and 6 denote three stacked round fixing plates, functioning as bottom, middle and top plate respectively. The plates 2, 4 resp. 6 have a hole 8, 10 resp. 12, surrounded by a ring 14, 16 resp. 18. The holes together define a central hole 20. Each ring has an inner edge I adjacent to the hole and an outer edge O at its outside.

Cable 22 forms a loop (not fully shown) outside the stack of plates. This loop can e.g. surround bone parts to be fixed. One end of cable 22 runs along a first continuous trajectory in which parts (a) to (d) are distinguished. Herein part (a) runs underneath the stack along the ring of the bottom plate 2 from its outer edge O to its inner edge I, part (b) runs through the central hole 20 along the inner edge I of the rings 14, 16 and 18 of the bottom plate 2, the middle plate 4 and the top plate 6 resp. to a position above and outside the stack, part (c) runs over the ring 18 of the top plate from its inner edge I to its outer edge O, part (d) runs along the outer edges O of one (case I), two (case (II) or all three (case III) of the rings of top plate 6 only, top plate 6 and middle plate 4 or top plate 6, middle plate 4 and bottom plate 2 resp. to a position, in case (I), at the level of the gap 24 between the top and the middle plate, in case (II), at the level of the gap 26 between the middle and the bottom plate and in case (III), below the stack. A section (e) of the second trajectory runs through gap 24, a section (f) through gap 26 and a section (h) runs along the bottom side of ring 14 of the bottom plate (g) from its outer edge O to its inner edge I. In each of the three cases (i) to (III) part (d) will connect to the corresponding section (e), (f) resp. (g) of the second trajectory. The required connecting sections to make the second trajectory continuous are not shown in FIG. 1 but they will be in the following Figures, showing various embodiments.

In FIG. 2 an embodiment of case (II) is shown. Part (d) ends at the level of gap 26 and the cable end 28 runs further as section (f) from the second trajectory through gap 26. Connecting section (i) runs upward through the central hole 20 and connects (f) to section (e) running through gap 24 bringing the cable outside the stack to be grabbed and tensioned. The other free end 30 of cable 22 follows a corresponding trajectory.

In FIG. 3 cable end 28 of cable 22 follows a first trajectory (a), (b), (c), (d), according to case III, wherein (d) ends below the stack and connects to section (g) of the second trajectory. The second trajectory then comprises the sections (e), (f), (g) that run as in FIG. 1, and further comprises section (h), which runs over the top side of the ring 18 of the top plate 6. The second trajectory is made a continuous trajectory by the connecting sections (i), (j) and (k), wherein (i) runs in an upward direction through the center hole along the inner edges I of the three plates and connects section (g) to section (h), wherein connecting section (j) runs in a downward direction along the outer edges O of the rings of top plate 6 and middle plate 4 and connects section (h) to section (f) and wherein (k) runs in an upward direction through the hole 10 along the inner edge I of ring 16 of middle plate 4 and connects section (f) to section (e) thus completing a continuous second trajectory (g)-(i)-(h)-(j)-(f)-(k)-(e). This trajectory ends with the cable end 28 running as section (e) from the central hole in an outward direction bringing the cable end 28 outside the stack.

The other end 30 of cable 22 follows a similar trajectory but mirrored with respect to the center of the holes. The ends 28 and 30 can be handled to be drawn and tension the cable 22, in particular around bone parts to keep these fixed with respect to each other.

In FIG. 4 an alternative trajectory is applied following the path (a)-(b)-(c)-(d)-(g)-(i)-(h)-(j)-(e)-(k)-(f). Connecting section (i) now runs in an upward direction, (j) runs in a downward direction and (k) also runs downward. Last section (f) brings cable end 28 outside the stack, to be grabbed and tensioned. The other free end 30 of cable 22 follows a corresponding trajectory.

In this embodiment, the surface of ring 16 of middle plate 4 that faces the bottom plate 2 a ridge 40 is present. On the surface of ring 14 of bottom plate 2 facing the middle plate 4 a groove 42 is present, in position and dimensions matching with ridge 40. Thus the last section of the total trajectory of the working end of cable 22 will be clamped in the groove by the ridge when the cable is tensioned. Details of ridge and groove are given in FIGS. 7 and 8.

In FIG. 5 a further alternative trajectory is applied following the path (a)-(b)-(c)-(d)-(g)-(i)-(e)-(j)-(h)-(k)-(f). Connecting section (i) runs in an upward direction, (j) also runs in an upward direction and (k) runs downward. Last section (f) brings cable end 28 outside the stack, to be grabbed and tensioned. The other free end 30 of cable 22 follows a corresponding trajectory. Also in this embodiment a co-operating groove-ridge combination is shown.

In FIG. 6 a further alternative trajectory is applied following the path (a)-(b)-(c)-(d)-(g)-(i)-(h)-(j)-(f)-(k)-(e). Connecting section (i) runs in an upward direction, (j) runs in a downward direction and (k) runs upward. Last section (e) brings cable end 28 outside the stack, to be grabbed and tensioned. The other free end 30 of cable 22 follows a corresponding trajectory. Also in this embodiment a co-operating groove-ridge combination is shown.

In FIG. 7a 750 is a circular fixing plate consisting of a ring 752, surrounding a hole 754. On the surface 756 of ring 752 an also circular ridge 758 is present, the centerline of which coincides with the centerline of ring 752.

In FIG. 7b on ring 752 a ridge 758 is present having the shape of a triangle having a top angle of 90° and a rounded top 760.

In FIG. 8a 850 is a circular fixing plate consisting of a ring 852, surrounding a hole 854. In the surface 856 of ring 852 an also circular groove 862 is present, the centerline of which coincides with the centerline of ring 852.

In FIG. 8b in ring 852 a groove 862 is present having the shape of a triangle having a top angle of 90° and matching with the ridge 758 of FIG. 7a.

1. Fixing device, comprising a surgical cable having a first and a second free end and at least three fixing plates positioned in a stack as a top plate, a middle plate and a bottom plate, leaving a gap between two adjacent plates, each fixing plate having a ring surrounding a central hole, the rings having an inner edge adjacent to the hole and an outer edge along a circumference of the fixing plate, wherein the holes in the stacked position at least partly overlap each other thus forming a central hole in the stack, and wherein at least one working end of the cable follows
(1) a first continuous trajectory starting from outside the stack running consecutively
(a) underneath the stack along the ring of the bottom plate from its outer edge to its inner edge,
(b) through the central hole along the inner edge of the rings of the bottom plate, the middle plate and the top plate respectively to a position above and outside the stack,
(c) over the ring of the top plate from its inner edge to its outer edge,
(d) along the outer edges of (I) the rings of the top plate, (II) of the top and the middle plate or (III) of the top, the middle and the bottom plate to a position, in case (I), at the level of the gap between the top and the middle plate, in case (II), at the level of the gap between the middle and the bottom plate and in case (III), below the stack,
(2) a second continuous trajectory, comprising in non-prescribed order and directions at least a section (e) through the gap between the top and the middle plate and a section (f) through the gap between the middle and the bottom plate, the second trajectory starting in case (I) with section (e), in case (II) with section (f) and in case (III) with a section (g) running along the bottom side of the ring of the bottom plate the second trajectory further comprising one or more connection sections, running through at least a part of the central hole or outside the outer edge of one or more of the rings, to make the trajectory continuous.

2. Fixing device according to claim 1 wherein the second trajectory ends with a last section being one of the sections (e) or (f).

3. Fixing device according to claim 2, wherein the cable in the last section runs through the corresponding gap from the inner edge to the outer edge of the rings adjacent to that gap bringing the free end from the central hole to outside the stack.

4. Fixing device according to claim 1, wherein the first trajectory runs in the order (a), (b), (c), (d).

5. Fixing device according to claim 4, wherein the second trajectory further comprises a section (h) running over the topside of the ring of the top plate.

6. Fixing device according to claim 1, wherein the other free end of the cable also follows one of the first and second trajectories.

7. Fixing device according to claim 1, wherein the other free end is fixed to a tensioning device that is connected to the fixing plates.

8. Fixing device according to claim 1, wherein in one of the rings adjacent to a gap in its surface facing the gap a continuous groove is present running between the outer edge and the inner edge of said ring and in the other ring adjacent to the gap in its surface facing the gap a ridge is present matching with said groove.

9. Fixing device according to claim 8 wherein the groove and matching ridge are present in the plates through which the last part of the second trajectory runs.

10. Method for tying together objects, in particular for fixing bone parts comprising the steps of applying a fixing device according to claim 1 around the bone parts to be fixed, followed by drawing the free ends of the cable to tension the cable around the bone parts to the tension required to fix the bone parts.

11. Method according to claim 10, wherein a bar is inserted between the fixing plates before the cable is tensioned and removed after the cable has been tensioned.

12. Method for tying together objects, in particular for fixing bone parts comprising the steps of applying a fixing device according to claim 7 around the bone parts to be fixed, followed by drawing said one end of the cable to tension the cable around the bone and then tensioning the cable to the tension required to fix the bone parts by means of the tensioning device.

13. Set of at least three fixing plates, each fixing plate having a ring surrounding a central hole, the rings having an inner edge adjacent to the hole and an outer edge along a circumference of the fixing plate, and a surgical cable fitted for constructing a fixing device according to claim 1.

14. Fixing plate, having a ring surrounding a central hole, the ring having an inner edge adjacent to the hole and an outer edge along a circumference of the fixing plate, prepared for application in a fixing device according to claim 1.

15. Fixing plate, having a ring surrounding a central hole, the ring having an inner edge adjacent to the hole and an outer edge along a circumference of the fixing plate, prepared for application in the method of claim 10.

16. Surgical cable prepared for application in a fixing device according to claim 1.

17. Surgical cable prepared for application in the method of claim 10.

18. Set of at least three fixing plates, each fixing plate having a ring surrounding a central hole, the rings having an inner edge adjacent to the hole and an outer edge along a circumference of the fixing plate, and a surgical cable prepared for application in the method of claim 10.

19. Set of two fixing plates, each fixing plate having a ring surrounding a central hole, the rings having an inner edge adjacent to the hole and an outer edge along a circumference of the fixing plate, in the surface of the ring of one plate a continuous groove being present running between the outer edge and the inner edge of said ring and in the surface of the ring of the other plate a ridge being present matching with said groove such that in a stacked position of the two rings the ridge fits in the groove.

20. Set of three fixing plates, each fixing plate having a ring surrounding a central hole, the rings having an inner edge adjacent to the hole and an outer edge along a circumference of the fixing plate, in the surface of both sides of the ring of a first of the plates a groove or a ridge being present running between the outer edge and the inner edge of said ring and in the surface of at least one side of a second of the three rings a groove or ridge matching with the ridge or groove on one side of the first plate is present and in the surface of at least one side of a third of the three rings a groove or ridge matching with the ridge or groove on the other side of the first plate is present, such that in a stacked position of the three rings said ridges fit in said grooves.
